(19) United States
(12) Patent Application Publication
(10) Pub. No.: US 2021/0401846 A1
(43) Pub. Date: Dec. 30, 2021

(54) MEDICAMENT, COMPOUND, COMPOSITION AND METHOD FOR TREATING HERPES

(71) Applicant: Napoleon Savescu, Long Island City, NY (US)

(72) Inventor: Napoleon Savescu, Long Island City, NY (US)

(21) Appl. No.: 17/347,272

(22) Filed: Jun. 14, 2021

(30) Foreign Application Priority Data
Jun. 29, 2020 (CA) 3085078

(51) Int. Cl. A61K 31/522

(52) U.S. Cl. CPC A61K 31/522

(57) ABSTRACT

An improved medicament, method and use for treating genital herpes in men (GHM) including a medicinal composition, which can be self-administered and maintained for a prescribed time. The medicinal composition can comprise one of an erectile dysfunction compound or medicament that can increase a genital amount of the blood flow, in combination with an anti-herpetic compound or medicament that can increase a large amount of blood flow specifically in the infected genital area. The result, of using the above medicament composition, not only achieved the disappearance of the local, genital herpetic infection, but also disappearance of the recurrence in the specific area. The medicinal composition can also include other compounds, additives, herbal extracts and/or carriers.

### CROSS-REFERENCE TO RELATED APPLICATION

The present application claims priority under 35 U.S.C. 119(a) to Canadian (CA) patent application number 3,085,078 filed on Jun. 29, 2020, which CA patent application 3,085,078 is incorporated herein by reference in its entirety.

### BACKGROUND OF THE INVENTION

### Technical Field

The present invention relates to a medicament, compound, composition and method for treating herpes. The compound, composition and method of the present technology has particular utility in connection with treating genital herpes in men (GHM), genital cold sores, rashes, skin conditions or symptoms resulting from GHM.

### Description of the Background Art

Genital sores in men, also known as blisters, are clusters of small blisters on the skin of the penis. They are called sores or blisters because they often occur during sex. The sores are small, painful, fluid-filled blisters on the skin of the penis. The skin around the blisters is often red and inflamed. The blisters can break open, weep a clear fluid, and then scab over after a few days.

The sores are caused by a virus known as herpes simplex type II (HSV-2) that attacks the skin of the penis and nervous system. HSV-1 is different from herpes simplex type II (HSV-2), which is the virus that causes the sexually transmitted disease known as genital herpes. Herpes simplex virus type 2 is usually responsible for genital herpes. However, either type of the herpes virus can cause sores on the genitals.

There are two types of herpes simplex virus:

Type 1 herpes simplex virus is the usual cause of cold sores around the mouth. It also causes more than half (50%) of cases of genital herpes in men.

Type 2 herpes simplex virus usually only causes genital herpes. It can sometimes cause cold sores.

Genital herpes is usually passed on by skin-to-skin contact with the affected area of someone who is already infected with the virus. The moist skin that lines the genitals of the men is the most susceptible to infection. This means that the virus is most commonly passed on by having vaginal, anal or oral sex, or just close genital contact with an infected person. For example, if someone has a cold sore around your mouth, by having oral sex, he may pass on the virus that causes genital herpes in men.

Herpes simplex virus can also enter through a cut or break in the ordinary skin on other parts of the body. In this way the virus can sometimes affect fingers, hands, knees, etc., if they are in contact with another person's infected area. It is called a whitlow when it is on the fingers.

After the first episode of sores/blisters, the herpes virus lies dormant in the nerves or skin around the original area until something sets the virus off into another eruption. Herpes simplex virus can be reactivated in response to various mechanic stimuli like, for example but not limited to, sex.

Genital sores/blisters are contagious and can be spread by direct skin-to-skin contact or by saliva.

HSV 1 and HSV 2 infections pose very serious health threats and can cause: blindness, increased cancer risk, aseptic meningitis and encephalitis, neonatal deaths, viremia; etc.

The devastating effects of this disease go well beyond the medical scope of human suffering. HSV is responsible for serious psychological and emotional distress as well as substantial economic loss to the nation and the world.

GHM is an infection of a part of the genital area with the herpes simplex virus, usually acquired through having sex. After an initial infection, the virus lies within the nerve root and skin of the penis because recurring symptoms from time to time.

It is usual that a person catches the infection the first time, from sexual contact. The person will not necessarily develop any symptoms at that point; indeed most people do not. The virus then retreats up a nerve, and from time to time is found back on the skin around the genital area. When the virus is active on the skin the person may still have no symptoms at all, or may have a painful rash. That means when the person first had the symptoms they are not necessarily when the person caught the infection.

The person might have caught it years ago and only just developed symptoms, or may be having a recurrence of the initial infection.

What is more, the person's partner might have caught it years ago and may have passed it on without even knowing it. Or the person's partner may have caught it years ago and had a recurrence, which has resulted in the person getting the infection.

But the priority is having treatment and avoiding passing it on to others, rather than trying to find somewhere to point the finger of blame.

As above, the person may never know of having the infection. Lots of people are infected with the herpes simplex virus without ever realizing. The most common symptom is a painful rash, which looks like one or more painful blisters or ulcers on the skin of genital area in men. The rash or pain can be around the penis or anus.

Usually the first time the person gets the rash is the worst. Recurring symptoms tend to be less severe.

Typical scenarios if the person thinks they have been infected are:

Seeing a primary care physician to confirm the diagnostic.

Get treatment.

Advising the person's partner(s) to get tested.

Some ways to avoid getting genital herpes or reduce the risk are:

Using condoms, which may not completely protect against herpes but they reduce the risk.

Having fewer sexual partners.

Avoid sex with anybody with active genital herpes (visible genital sores or blisters).

A partner with recurrent herpes can take an antiviral medicine to reduce risk.

Unfortunately, once you have had the infection, there is no way of completely getting rid of it from your body. So the person could be infectious on and off without necessarily knowing it. There is also no guaranteed way of avoiding genital herpes. However, there are some measures, which can help in avoiding getting herpes, or avoid passing it on to others.

Some symptoms of genital herpes can be, the first time the person is infected with genital herpes simplex it is called the primary infection. This may, or may not, cause symptoms (described below). Following a primary infection, the virus is not cleared from the body but lies inactive (dormant) in a nearby nerve. In some people, the virus activates from time to time and travels down the nerve to the penis skin. This causes recurrent symptoms of genital herpes in men.

It is common not to develop any symptoms. Most people never develop any symptoms when they are infected with the virus. At least 8 in 10 people (80%) with genital herpes simplex virus do not know that they are infected, or they only have a short bout of very mild symptoms, which is not recognized as genital herpes. For example, just a slight area of itch or a small red area on the skin of the penis, which soon goes. In such people, the virus stays inactive in the root of a nerve that supplies the genitals, but never causes recurrent episodes of symptoms. However, even people who do not develop symptoms may, on occasions, have virus in their genital area and therefore be infectious to sexual partners. In fact, this is how many genital herpes simplex infections are passed on.

A first episode of symptoms can include, the person may feel generally unwell with a mild fever and aches and pains. Groups of small, painful blisters then appear on the skin of the person's penis. They tend to erupt in crops over 1-2 weeks. The blisters soon burst and turn to shallow, sore ulcers.

The glands in the person's groin may swell and feel like lumps at the top of your legs. It is common sometimes to have pain when the person passes urine.

Sometimes less typical symptoms occur. For example, the person may just have a small raw area, one or two small ulcers, or just an area of irritation with nothing to see. Sometimes symptoms last just a few days.

Sometimes a first episode of symptoms appears months or years after being first infected. This is why a first episode of symptoms can occur during a current faithful sexual relationship. The person may have been infected months or years ago from a previous sexual partner who did not realize that they were infected.

It is not clear why some infected people develop symptoms, some do not and some have a first episode of symptoms months or years after first being infected. It may be something to do with the way the immune system reacts to the virus in different people.

Recurring episodes of symptoms can include after the first episode, further episodes of symptoms occur in some people from time to time. This is called recurrent infection. It is not clear why the dormant virus erupts from time to time. Recurrences tend to be less severe and shorter than the first episode. It is more usual to have 7-10 days of symptoms with a recurrence, unlike the longer phase of symptoms that may occur during the first episode. Most people do not develop a fever and do not feel particularly unwell during a recurrence. A tingling or itch in your genital area for 12-24 hours may indicate a recurrence is starting. The time period between recurrences is variable.

Recurrences tend to become less frequent over time. In people who have recurrences, their frequency can vary greatly. Some people have six or more a year. For others it is less frequent than this. On average, people tend to have 1 to 4 recurrences per year during the first two years after the first episode. Some people do not have recurrences at all after a first episode of symptoms. Some people can identify some things that may trigger a recurrence.

If someone suspects that may have genital herpes or any other sexually transmitted infection then must see, as soon as possible, a primary care physician or to contact a local medical clinic. A doctor or nurse can swab a blister to obtain a small sample to send it to the lab. This can confirm the infection is due to the herpes simplex virus. It may also find out which type of herpes virus has caused the infection.

Tests to look for other sexually transmitted infections may also be done at the same time. Sometimes a blood test is done as well. This determines whether you have had a herpes infection in the past, or whether this is the first time. It can also tell which type of herpes simplex virus it is.

After diagnosis, the classical treatment for genital herpes can include general measures that may help to ease symptoms when they occur, including:

Administration of painkillers to ease pain.

A numbing (anesthetic) ointment, for example lidocaine 5% gel, apply on affected area may relieve itching or pain. It is noted however some people may be sensitive (allergic) to anesthetic ointments, and the ointment then makes skin symptoms worse. Applying Vaseline may be a helpful alternative to anesthetic ointment.

Ice wrapped in a tea towel (an ice pack) placed over the genital sores for 5-10 minutes may be soothing. Do not put ice directly on to skin, as this may cause an ‘ice burn’.

Do not use scented soaps, bubble bath, etc., as these may irritate the skin of the penis. Gentle cleaning of the sores with just cotton wool and plain or salt water is best. Gentle drying with a hairdryer on its lowest setting may be more comfortable than with a towel.

When resuming sexual activity after an episode has cleared, a lubricant may help, as some people find the friction of having sex may trigger a recurrence.

The person will not pass on this virus through using towels, facecloths, toilets or swimming pools.

The person should avoid having sex until the sores and blisters have cleared and/or you have seen a doctor for follow-up.

It is best to be honest and tell your sexual partner if you have been diagnosed with genital herpes. If they have not got the infection, the doctor will explain ways to reduce the chances of passing it on to them. The doctor will also help explain that because of the way the virus works, it is not possible to tell how long ago you acquired the infection. Sometimes people are scared to tell their partners in case their partner thinks they have been unfaithful. Or it may be that they are worried their partner has been unfaithful and given them the infection. But because there is often a long time lag, this is often not the case. The doctor will help you with these worries.

Classical antiviral medication does not clear the virus from the body. It works by stopping the virus from multiplying. Antiviral medicines include Aciclovir (Acyclovir, Acycloguanosine), Famciclovir (Famcyclovir, Famvir®) and Valaciclovir (Valacyclovir). Antiviral medication is most useful for a first episode of symptoms. It reduces the severity and duration of symptoms if it is started within five days of symptoms starting. A five-day course of treatment is usual but may be extended by a few days if blisters are still forming.

Antiviral medication may not be needed to treat recurrences. This is because symptoms are often much milder than the first episode and usually last just a few days. However, if the person tends to have bad symptoms during recurrences, then a course of medication can be useful. To reduce the duration and severity of a recurrence, start the medication as soon as symptoms begin. Some doctors prescribe antiviral medication that you can keep at home and can start at the first sign of a recurrence. Starting treatment early can help to reduce the severity of your symptoms, but till today not to cure/solve the problem.

If the person has frequent recurrences, an option is to take antiviral medication every day. In most people who take medication every day, the recurrences are either stopped completely, or their frequency and severity are greatly reduced. This is called suppressive treatment.

There is not yet a vaccine to protect against the herpes virus. There are a number of things to consider which may reduce your risks of getting genital herpes or of passing it on to others.

These include:

Consider the use of condoms always, even in settled relationships. This is because a person can carry the herpes virus for a very long time and pass it on without ever being aware of it. Condoms do not completely protect against herpes but they reduce the risk.

The more sexual partners someone has, the more the risk of picking up any sexually transmitted infection, including genital herpes. So avoiding having too many partners will cut down your risk.

Avoid having sex with somebody with an active genital herpes infection (somebody with visible genital sores or blisters).

Also avoid intimate contact with a person who has a cold sore.

If you have an active genital herpes infection yourself, avoid having sex with anyone else in order to prevent passing it on.

If one partner finds out they have herpes, it is wise to tell the other. This can reduce transmission rates.

If a person knows they have recurrent herpes, taking a regular antiviral medicine can reduce the risk of passing on the virus.

Herpes simplex virus is very contagious when blisters are present. There is a high chance of passing on the virus if you have sex. You should not have sex from the time symptoms first start until they are fully over. If you do have sex, using a condom may not fully protect against passing on the virus, as the condom only protects the area that is covered.

It is less likely that you will pass the virus on when you have sex. However, some virus will be present on the genital skin surface from time to time, although infrequently. So, there is still a small chance that you may pass on the virus when you have sex when you do not have symptoms.

It is best to discuss things with your sexual partner. Using a condom each time you have sex is thought to reduce the chance further. However, using a condom cannot completely stop the chance of passing on the virus.

Taking antiviral medication long-term to prevent recurrences (suppressive treatment) also reduces the risk of passing on the virus. However, very few people need to take this treatment all the time. It is noted that if your sexual partner already has the same virus then you cannot re-infect each other.

Currently about one out of five people (20%) in the United States has genital herpes. That is more than 50 million people in the United States who have genital herpes. 85% of people with genital herpes do not know they have it. That is 42 million Americans who are unaware they have genital herpes. Genital herpes is the most prevalent viral STD.

Herpes Statistics in Specific Populations:

Genital herpes affects more Black than White Americans: 39.2% of the overall Black population, with 48% of Black women affected. The largest increasing population is white teenagers. One in four American teenagers has an STD. 50-75% of unmarried American women between 45 and 50 have genital herpes. Globally, an estimated 536 million people are infected.

Note: Not all genital herpes statistics are consistent from study to study. Most say 25% of American women have the virus, and 20% of American males. Other studies show slightly lower numbers. The studies with slightly lower numbers refer to people from 14 to 49, while the higher number studies are based on all people over 12. This is the reason, at least to the best of my knowledge.

Center for Disease Control (CDC) Analysis of National Herpes Prevalence indicated that some recent media reports have questioned the accuracy of CDC's latest report on national herpes prevalence (herpes simplex virus type 2, or HSV-2). HSV-2 is a lifelong and incurable infection that can cause recurrent and painful genital sores and can make those infected with the virus two-to-three times more likely to acquire HIV, the virus that causes AIDS.

The latest HSV-2 data—announced at CDC's National STD Conference in Atlanta on Mar. 9, 2010, and published today in CDC's Morbidity and Mortality Weekly Report (MMWR)—indicates that overall national HSV-2 prevalence remains high (16.2%) and that the disease continues to disproportionately burden African-Americans (39.2% prevalence), particularly black women (48.0% prevalence), who face a number of factors putting them at greater risk, including higher community prevalence and biological factors that put women of all races at greater risk for HSV-2 than men.

While these findings may be surprising to some—they are, in fact, an accurate representation of the prevalence of HSV-2 infection in these populations and are consistent with prior data on the scope of the problem. CDC stands firmly behind these statistics and the methodology used to develop them. The data come from the National Health and Nutritional Examination Survey (NHANES), a nationally representative survey that has been continuously conducted by the National Center for Health Statistics since the early 1960s. The survey is one of the most reliable sources of data on American health available today, providing representative data on dozens of major diseases, including cardiovascular disease and diabetes.

This analysis provides data on the percentage of Americans who are infected with HSV-2, or genital herpes, based on the number of people who tested positive for HSV-2 antibodies. These antibodies are only present if an individual is infected with the virus. While not all infected individuals develop symptoms of HSV-2, they can pass this lifelong infection on to others without knowing it.

CDC published these data to ensure that the general public, along with those at highest risk, have the information they need to take the necessary steps to protect themselves, their partners and their children. This latest analysis emphasizes that we cannot afford to be complacent about this and other sexually transmitted infections. Any information that minimizes the severity of this public health challenge ultimately hinders efforts to prevent STDs in this country.

As shown in FIG. 1, Herpes Simplex Virus Type 2—Sero prevalence Among Non-Hispanic Whites and Non-Hispanic Blacks by Sex and Age Group, National Health and Nutrition Examination Survey (NHANES), 1988-1994, 1999-2002, 2003-2006, and 2007-2010. SOURCE: Fanfair R N, Zaidi A, Taylor L D, et al. Trends in seroprevalence of herpes simplex virus type 2 among Non-Hispanic Blacks and Non-Hispanic Whites aged 14 to 49 years—United States, 1988 to 2010. Sex Transm Dis 2013; 40(11):860-4.

Therefore, a need exists for a new and improved compound, composition and method for treating genital herpes. In this regard, the present invention substantially fulfills this need. In this respect, the compound, composition and method for treating genital herpes according to the present technology substantially departs from the conventional concepts and designs of the prior art, and in doing so provides an apparatus primarily developed for the purpose of treating genital herpes.

### SUMMARY OF THE INVENTION

In view of the foregoing disadvantages inherent in the known types of herpes treatment compounds now present in the prior art, the present invention provides an improved medicament, compound, composition and method for treating genital herpes, and overcomes the above-mentioned disadvantages and drawbacks of the prior art. As such, the general purpose of the present invention, which will be described subsequently in greater detail, is to provide a new and improved medicament, compound, composition and method for treating genital herpes and method which has all the advantages of the prior art mentioned heretofore and many novel features that result in a medicament, compound, composition and method for treating genital herpes which is not anticipated, rendered obvious, suggested, or even implied by the prior art, either alone or in any combination thereof.

In some aspects, the present technology provides novel combination of known agents for use in the treatment of genital herpes. The combinations advantageously display synergistic effects greater than would be predicted based on the additive levels of treatment displayed by the individual agents when used alone. In addition, the combinations can be self-administered.

According to one aspect, the present technology can include a medicament for use in treating a viral infection, the medicament comprising a combination composition including an anti-herpetic compound or a pharmaceutically acceptable salt thereof and an erectile dysfunction compound or a pharmaceutically acceptable salt thereof.

According to another aspect, the present technology can include a use of an anti-herpetic compound or a pharmaceutically acceptable salt thereof and an erectile dysfunction compound or a pharmaceutically acceptable salt thereof in the preparation of a medicament for treating genital herpes in men.

According to yet another aspect, the present technology can include a use of an anti-herpetic compound or a pharmaceutically acceptable salt thereof and an erectile dysfunction compound or a pharmaceutically acceptable salt thereof in the preparation of a medicament for treating genital herpes in men, wherein the medicament comprises a combination composition of approximately 5 mg to 50 mg of the erectile dysfunction compound and approximately 500 mg to 1000 mg of the anti-herpetic compound per dose.

In some or all embodiments, the viral infection can be herpes.

In some or all embodiments, the herpes can be herpes simplex type II.

In some or all embodiments, the anti-herpetic compound can be selected from the group consisting of valacyclovir and famciclovir, or pharmaceutically acceptable salt thereof, and wherein the erectile dysfunction compound can be selected from the group consisting of sildenafil, tadalafil, avanafil and vardenafil, or pharmaceutically acceptable salt thereof.

In some or all embodiments, the combination composition can include approximately 5 mg to 50 mg of the erectile dysfunction compound and approximately 500 mg to 1000 mg of the anti-herpetic compound.

In some or all embodiments, the erectile dysfunction compound and the anti-herpetic compound can each in the form of one or more solid dosage forms, tablets, capsules or in liquid form.

In some or all embodiments, the erectile dysfunction compound can be for use once a day for 14 days.

Some or all embodiments can include wherein 50 mg of the erectile dysfunction compound can be for use once a day for an initial 3 days of the 14 days, and then 25 mg of the erectile dysfunction compound is for use once a day for a remaining 11 days of the 14 days.

In some or all embodiments, the anti-herpetic compound can be for use at least three times a day for an initial 3 days of the 14 days, two times a day for a next 3 days of the 14 days, and once a day for a next 8 days of the 14 days.

Some or all embodiments can include wherein 1000 mg of the anti-herpetic compound can be for use three times a day for the initial 3 days of the 14 days, then 1000 mg of the anti-herpetic compound can be for use twice a day for the next 3 days of the 14 days, and then 1000 mg of the anti-herpetic compound can be for use once a day for the next 8 days of the 14 days.

In some or all embodiments, the anti-herpetic compound can be valacyclovir and the erectile dysfunction compound can be sildenafil.

Some or all embodiments can include wherein 10 mg of the erectile dysfunction compound can be for use once a day for an initial 3 days of the 14 days, and then 5 mg of the erectile dysfunction compound can be for use once a day for a remaining 11 days of the 14 days.

In some or all embodiments, the anti-herpetic compound can be for use at least three four times a day for an initial 3 days of the 14 days, three times a day for a next 2 days of the 14 days, two times a day for a next 4 days of the 14 days, and once a day for a next 5 days of the 14 days.

Some or all embodiments can include wherein for the initial 3 days of the 14 days 500 mg of the anti-herpetic compound can be for use three times a day and 1000 mg of the anti-herpetic compound can be for use once a day, wherein for the next 2 days of the 14 days 500 mg of the anti-herpetic compound can be for use three a day, wherein for the next 4 days of the 14 days 500 mg of the anti-herpetic compound can be for use twice a day, and wherein for the next 5 days of the 14 days 500 mg of the anti-herpetic compound can be for use once a day.

In some or all embodiments, the anti-herpetic compound can be famciclovir and the erectile dysfunction compound is tandalafil.

In some or all embodiments, the medicament can be presented as a pharmaceutical pack having the tablets or capsules of the anti-herpetic compound and the erectile dysfunction compound, the pack including instructions to a user to take one or more of the tablets or capsules for fourteen days.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of the invention, but nonetheless illustrative, embodiments of the present invention when taken in conjunction with the accompanying drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention.

An even further object of the present invention is to provide a new and improved medicament, compound, composition and method for treating genital herpes that has a low cost of manufacture with regard to both materials and labor, and which accordingly is then susceptible of low prices of sale to the consuming public, thereby making such medicament, compound, composition and method for treating genital herpes economically available to the buying public.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is representative charts showing trends in seroprevalence of herpes simplex virus type 2 among Non-Hispanic Blacks and Non-Hispanic Whites aged 14 to 49 years.

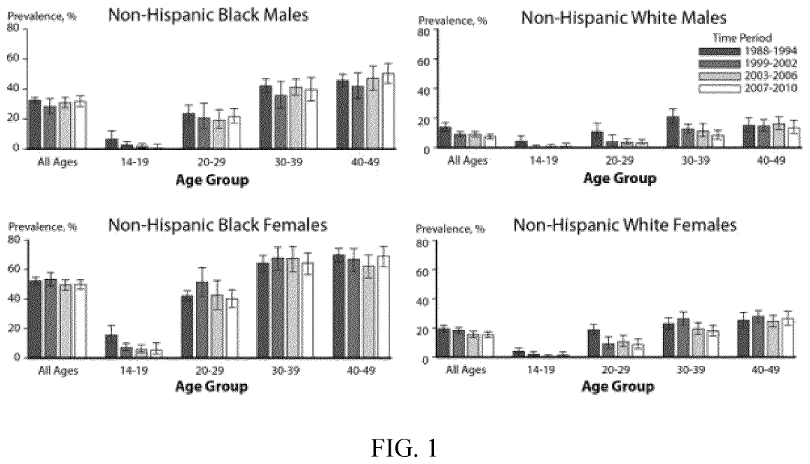

### DETAILED DESCRIPTION OF THE INVENTION

The present technology includes a novel medicament, compound, composition and method or use including the combination of a generic or non-generic erectile dysfunction (ED) compound or medication and a generic or non-generic anti-herpetic compound or medication. The present compound can be self-administered and maintained for a prescribed time.

The preparation of pharmacologically suitable compositions or the utilization of compounds for use as medicaments is well known to those of skill in the art. Typically, such compositions are prepared either as liquid solutions or suspensions, however solid dry forms such as tablets, pills, powders and the like are also contemplated. The liquid may be an aqueous liquid. Solid forms suitable for solution in, or suspension in, liquids prior to administration may also be prepared. The preparation may also be emulsified. The active ingredients of each compound may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. Some examples of materials which can serve as acceptable carriers or formulation components include, but are not limited to, ion exchangers, alumina, aluminum stearate, lecithin, various proteins, buffer substances (e.g. phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; and phosphate buffer solutions, as well as other non-toxic compatible lubricants such as sodium lauryl sulfate and magnesium stearate; coloring agents; releasing agents; coating agents; sweetening and flavoring agents, etc. Preservatives and antioxidants can also be present in the composition, according to the judgment of the formulator.

The medicament composition of the present technology may contain any such additional ingredients so as to provide the composition in a form suitable for administration.

The final amount of active agents described herein in the formulations may vary. However, in general, the amount or each is from about 1% to about 99%.

The methods or uses of the present technology involve treating infections in a subject comprising using a medicament or using compounds to prepare a medicament to treat a subject with a therapeutically effective amount of at least one composition of the present technology. The methods or uses of the present technology may also involve identifying subjects or patients who might benefit from receiving therapy for any of these infections through administration of a composition or medicament comprising at least one of the compounds described herein. Generally a suitable subject or patient is identified by a health care professional or professionals using known tests, measurements, or criteria for either already having symptoms of an infection or being at risk of developing symptoms of an infection. A suitable treatment protocol is then developed. The methods or uses may also comprise one or more steps related to monitoring the effects or outcome of administration in order to evaluate the treatment protocol and/or to adjust the protocol as required or in a manner that is likely to provide more benefit, e.g. by increasing or decreasing doses of medication, or by changing the particular type of compound that is administered, or by changing the frequency of dosing or the route of administration, etc. While in some cases the improvement or lessening of symptoms (or the prevention of symptoms) that occurs may be complete, e.g. the functioning of the patient returns to or remains normal (as assessed in comparison to suitable control subjects or standardized values obtained therefrom), this need not always be the case. Those of skill in the art will recognize that even a lower level of improvement in symptoms may be highly beneficial to the patient, as may be the slowing of the progression or symptoms of the infection, even if a complete cure does not result.

The term “therapeutically effective amount” refers to an amount of a compound or composition effective to treat a disease, disorder, or infection in a subject. The therapeutically effective amount of the compound or composition may reduce and/or prevent or slow the progression to some extent of one or more of the symptoms associated with the disease, disorder, or infection.

The methods or uses of the present technology involve administering compositions or medicament comprising at least one (i.e. one or more) of the drugs/compounds disclosed herein to a patient in need thereof. The drugs may be administered simultaneously or sequentially; separately or together in the same formulation. The present invention thus also provides compositions which comprise the compounds as described herein, usually together with a pharmacologically suitable carrier or diluent. In some or all embodiments, one substantially purified compound is present in a composition; in other embodiments more than one compound is present, each compound being substantially purified prior to being mixed in the composition.

The compound compositions (preparations) or medicaments of the present technology may be administered by any of the many suitable means which are well known to those of skill in the art, including but not limited to: by injection, inhalation, orally, intravaginally, intranasally, by ingestion of a food or product containing the agent, topically, as eye drops, via sprays, etc. In exemplary embodiments, the mode of administration is orally or by injection. In addition, the compositions may be administered in conjunction with other treatment modalities such as other agents which are used to treat viral infections, microbial infections, sores or ulcers, examples of which include but are not limited to the administration of antibiotics, steroids, ointments, etc.

The dose of the compounds or medicaments and the timing and mode of administration may vary from individual to individual e.g. based on the type of infection; the stage of the r infection (if the infection is initial or reoccurring); the age, gender, weight, genetic background, and overall general health of the individual, etc., and is best determined by a skilled medical practitioner such as a physician. Frequency of administration generally ranges from 1 to 5 times per day for 14 days, although slow release formulations may permit administration daily or every few days.

In some or all embodiments, the compounds or medicaments described herein can be used prophylactically, e.g. they are used with people who have not yet exhibited symptoms of the infection, or simply those who are at risk due to other factors such as exposure to an infectious person. The compounds or medicaments may also be administered to individuals who are thought or deemed to be exhibiting early signs of infection or to be in early stages of infection. Those of skill in the art will recognize that, while complete remission of infection may be desirable, great benefit may also accrue if partial remission or slowing of infection progress is achieved.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

The presently disclosed subject matter now will be described more fully hereinafter. The presently disclosed subject matter may be embodied in many different forms and should not be construed as limited to the embodiments set forth herein; rather, these embodiments are provided as exemplary. Indeed, many modifications and other embodiments of the presently disclosed subject matter set forth herein will come to mind to one skilled in the art to which the presently disclosed subject matter pertains having the benefit of the teachings presented in the foregoing descriptions.

The ED compound increases blood flow to the genital area, and the anti-herpetic compound increases blood flow to the infected area of the genitals.

The present invention relates to medical treatment, and more particularly, the cure and disappearance forever of the recurrence of to the lesions on genital herpes in men: cold sores, rashes, skin conditions, or local symptoms resulting from this infection.

The result, of using the above compounds, not only achieved the disappearance of the local, genital herpetic infection in the treated man, but also disappearance of the recurrence in the specific area. Not to mention the economic effect and personal increasing of the self-esteems in the affected men with genital herpes.

Desirably, the medicinal composition help to cure, the ulcerations/blisters—the infected regions of the penis, external symptoms and physical manifestations of the symptoms resulting from genital herpes in men.

A process to treat GHM an infection of a part of the genital area with the herpes simplex virus, usually acquired through having sex. After an initial infection, the virus lies within the nerve root, and skin of the penis cause recurring symptoms from time to time of the genital herpes in men and to cure cold sores, rashes, skin conditions/blisters, or symptoms resulting from genital herpes in men, comprising: providing an anti-herpes medicinal (AHM) compound or composition in combination with an ED medication.

The ED medicines that can be utilized in the present technology can include, but not limited to, a PDE5 inhibitor, avanafil, lodenafil, mirodenafil, sildenafil citrate (Revatio®, Viagra®, or analogs thereof, for example, actetildenafil, hydroxyacetildenafil, or dimethyl-sildenafil), tadalafil (Adcirca®, Cialis®), avanafil (Stendra®), vardenafil (Levitra®), udenafil, acetildenafil, thiome-thisosildenafil, alprostadil or testosterone replacement.

PDE5 inhibitors have also been studied for other clinical use as well, including cardiovascular and heart diseases. For example, because PDE5 is also present in the arterial wall smooth muscle within the lungs, PDE5 inhibitors have also been explored for lung diseases such as pulmonary hypertension and cystic fibrosis. Pulmonary arterial hypertension, a disease characterized by sustained elevations of pulmonary artery pressure, which leads to an increased incidence of failure of the right ventricle of the heart, which in turn can result in the blood vessels in the lungs become overloaded with fluid. PDE5 inhibitors have been found to have activity as both a corrector and potentiator of CFTR protein abnormalities in animal models of cystic fibrosis disease.

The AHM medicament that can be utilized in the present technology can include, but not limited to, Acyclovir (Zovirax®), Valacyclovir (Valtrex®), or Famciclovir (Famvir®).

The above combination provided disappearance of the skin lesions from genital area in men forever.

The ED medication is increasing the blood flow in the cavernous area of the penis, and in combination with AHM automatically will increase it, only locally, accordingly quit few times (otherwise, these increase would be in all our body with toxic side effects present).

The above combination, ED medication with AHM, will cure local skin lesions in the genital area of men.

### Example 1—Dacia Herpes Pack 1

Compound 1—Antiviral agent (Valacyclovir)+Compound 2—ED—Phospho Diesterase 5 inhibitor (Sildenafil)

Compound 1: Valtrex®/Valacyclovir (C₁₃H₂₀N₆O₄)

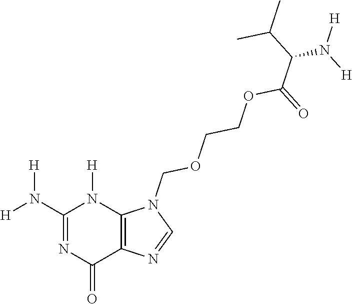 [SMILES: CC(C)[C@H](N)C(=O)OCCOCn1cnc2c(=O)nc(N)[nH]c21]

### Mechanism of Action:

Valacyclovir is the hydrochloride salt of the L-valyl ester of the antiviral drug acyclovir. Orally administered, valacyclovir is rapidly converted to acyclovir (C₈H₁₁N₅O₃) which inhibits viral DNA replication after further conversion to the nucleotide analog acyclovir triphosphate (C₈H₁₄N₅O₁₂P₃) by viral thymidine kinase, cellular guanyl cyclase, and a number of other cellular enzymes. Acyclovir triphosphate competitively inhibits viral DNA polymerase; incorporates into and terminates the growing viral DNA chain; and inactivates viral DNA polymerase. The greater antiviral activity of acyclovir against herpes simplex virus (HSV) compared with varicella-zoster virus (VZV) is due to its more efficient phosphorylation by HSV thymidine kinase.

Pharmacology from NCIt:

Valacyclovir is a nucleoside analogue antiviral agent and prodrug of acyclovir, which is used in therapy of herpes and varicella-zoster virus infections. Valacyclovir has been associated with rare instances mild, clinically apparent liver injury.

### Pharmacodynamics and Pharmacokinetics:

Absorption: Rapid

Distribution: Acyclovir is widely distributed throughout the body including brain, kidney, lungs, liver, spleen, muscle, uterus, vagina, and CSF

Protein binding: ˜14% to 18%

Metabolism: Hepatic; valacyclovir is rapidly and nearly completely converted to acyclovir and L-valine by first-pass effect; acyclovir is hepatically metabolized to a very small extent by aldehyde oxidase and by alcohol and aldehyde dehydrogenase (inactive metabolites)

Bioavailability: ˜55% once converted to acyclovir

Half-life elimination: Normal renal function: Adults: Acyclovir: 2.5-3.3 hours, Valacyclovir: ˜30 minutes; End-stage renal disease: Acyclovir: 14 to 20 hours; During hemodialysis: 4 hours

Excretion: Urine, primarily as acyclovir (89%); Note: Following oral administration of radiolabeled valacyclovir, 46% of the label is eliminated in the feces (corresponding to nonabsorbed drug), while 47% of the radiolabel is eliminated in the urine.

Compound 2: Viagra®/Sildenafil (Phospho Diesterase 5 inhibitors) (C₂₂H₃₀N₆O₄S)

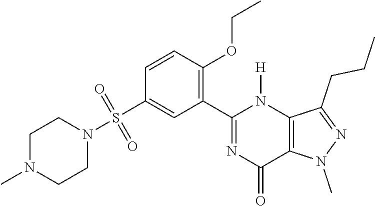 [SMILES: CCCc1nn(C)c2c(=O)nc(-c3cc(S(=O)(=O)N4CCN(C)CC4)ccc3OCC)[nH]c12]

### Mechanism of Action:

Erectile dysfunction: Does not directly cause penile erections, but affects the response to sexual stimulation. The physiologic mechanism of erection of the penis involves release of nitric oxide (NO) in the corpus cavernosum during sexual stimulation. NO then activates the enzyme guanylate cyclase, which results in increased levels of cyclic guanosine monophosphate (cGMP), producing smooth muscle relaxation and inflow of blood to the corpus cavernosum. Sildenafil enhances the effect of NO by inhibiting phosphodiesterase type 5 (PDE-5), which is responsible for degradation of cGMP in the corpus cavernosum; when sexual stimulation causes local release of NO, inhibition of PDE-5 by sildenafil causes increased levels of cGMP in the corpus cavernosum, resulting in smooth muscle relaxation and inflow of blood to the corpus cavernosum; at recommended doses, it has no effect in the absence of sexual stimulation. An exemplary pack dose for 2 weeks is shown in Table 1.

TABLE 1

| Day | Viagra ®/Sildenafil (mg) | Valtrex/Valacyclovir (mg) |
|---|---|---|
| 1 | 50 mg | 1000 + 1000 + 1000 |
| 2 | 50 mg | 1000 + 1000 + 1000 |
| 3 | 50 mg | 1000 + 1000 + 1000 |
| 4 | 25 mg | 1000 + 1000 |
| 5 | 25 mg | 1000 + 1000 |
| 6 | 25 mg | 1000 + 1000 |
| 7 | 25 mg | 1000 |
| 8 | 25 mg | 1000 |
| 9 | 25 mg | 1000 |
| 10 | 25 mg | 1000 |
| 11 | 25 mg | 1000 |
| 12 | 25 mg | 1000 |
| 13 | 25 mg | 1000 |
| 14 | 25 mg | 1000 |

### Example 2—Dacia Herpes Pack 2

Compound 1—Antiviral agent (famciclovir)+Compound 2—ED—Phospho Diesterase 5 inhibitor (Tadalafil)

Compound 1: Famvir®/famciclovir (C₁₄H₁₉N₅O₄)

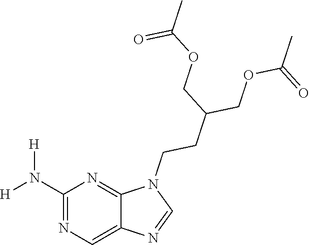 [SMILES: CC(=O)OCC(CCn1cnc2cnc(N)nc21)COC(C)=O]

### Mechanism of Action:

Famciclovir is a Herpes Simplex Virus Nucleoside Analog DNA Polymerase Inhibitor.

The mechanism of action of famciclovir is as a DNA Polymerase Inhibitor, and DNA Polymerase Inhibitor. The chemical classification of famciclovir is Nucleoside Analog.

Famciclovir is a diacetyl 6-deoxy prodrug analog of the antiviral agent penciclovir. Orally administered, famciclovir in vivo is converted to penciclovir triphosphate (C₁₀H₁₈N₅O₁₂P₃), which is active against the Herpes viruses, including herpes simplex 1 and 2 and varicella-zoster. This agent inhibits the replication of viral DNA by interfering competitively with DNA polymerase. (NCI04)

Famciclovir is a nucleoside analogue and antiviral agent used in therapy of herpes zoster and simplex virus infections. Famciclovir is associated with a low rate of mild-to-moderate serum ALT elevations during therapy, but has not been associated with instances of clinically apparent liver injury.

### Pharmacodynamics and Pharmacokinetics:

The absolute bioavailability of penciclovir is 77±8% as determined following the administration of a 500 mg famciclovir oral dose and a 400 mg penciclovir intravenous dose to 12 healthy male subjects.

Penciclovir concentrations increased in proportion to dose over a famciclovir dose range of 125 mg to 1000 mg administered as a single dose. Table 5 shows the mean pharmacokinetic parameters of penciclovir after single administration of Famvir® to healthy male volunteers.

Following oral single-dose administration of 500 mg famciclovir to 7 patients with herpes zoster, the AUC (mean±SD), Cmax, and tmax were 12.1±1.7 mcg hr/mL, 4.0±0.7 mcg/mL, and 0.7±0.2 hours, respectively. The AUC of penciclovir was approximately 35% greater in patients with herpes zoster as compared to healthy volunteers. Some of this difference may be due to differences in renal function between the 2 groups.

There is no accumulation of penciclovir after the administration of 500 mg famciclovir three times daily for 7 days.

Penciclovir Cmax decreased approximately 50% and tmax was delayed by 1.5 hours when a capsule formulation of famciclovir was administered with food (nutritional content was approximately 910 Kcal and 26% fat). There was no effect on the extent of availability (AUC) of penciclovir. There was an 18% decrease in Cmax and a delay in tmax of about 1 hour when famciclovir was given 2 hours after a meal as compared to its administration 2 hours before a meal. Because there was no effect on the extent of systemic availability of penciclovir, Famvir® can be taken without regard to meals.

Distribution:

The volume of distribution (Vdβ) was 1.08±0.17 L/kg in 12 healthy male subjects following a single intravenous dose of penciclovir at 400 mg administered as a 1-hour intravenous infusion. Penciclovir is <20% bound to plasma proteins over the concentration range of 0.1 to 20 mcg/mL. The blood/plasma ratio of penciclovir is approximately 1.

### Metabolism:

Following oral administration, famciclovir is deacetylated and oxidized to form penciclovir. Metabolites that are inactive include 6-deoxy penciclovir, monoacetylated penciclovir, and 6-deoxy monoacetylated penciclovir (5%, <0.5% and <0.5% of the dose in the urine, respectively). Little or no famciclovir is detected in plasma or urine. An in vitro study using human liver microsomes demonstrated that cytochrome P450 does not play an important role in famciclovir metabolism. The conversion of 6-deoxy penciclovir to penciclovir is catalyzed by aldehyde oxidase. Cimetidine and promethazine, in vitro inhibitors of aldehyde oxidase, did not show relevant effects on the formation of penciclovir in vivo].

### Elimination:

Approximately 94% of administered radioactivity was recovered in urine over 24 hours (83% of the dose was excreted in the first 6 hours) after the administration of 5 mg/kg radiolabeled penciclovir as a 1-hour infusion to 3 healthy male volunteers. Penciclovir accounted for 91% of the radioactivity excreted in the urine.

Following the oral administration of a single 500 mg dose of radiolabeled famciclovir to 3 healthy male volunteers, 73% and 27% of administered radioactivity were recovered in urine and feces over 72 hours, respectively. Penciclovir accounted for 82% and 6-deoxy penciclovir accounted for 7% of the radioactivity excreted in the urine. Approximately 60% of the administered radiolabeled dose was collected in urine in the first 6 hours.

After intravenous administration of penciclovir in 48 healthy male volunteers, mean±SD total plasma clearance of penciclovir was 36.6±6.3 L/hr (0.48±0.09 L/hr/kg). Penciclovir renal clearance accounted for 74.5±8.8% of total plasma clearance.

Renal clearance of penciclovir following the oral administration of a single 500 mg dose of famciclovir to 109 healthy male volunteers was 27.7±7.6 L/hr. Active tubular secretion contributes to the renal elimination of penciclovir.

The plasma elimination half-life of penciclovir was 2.0±0.3 hours after intravenous administration of penciclovir to 48 healthy male volunteers and 2.3±0.4 hours after oral administration of 500 mg famciclovir to 124 healthy male volunteers. The half-life in 17 patients with herpes zoster was 2.8±1.0 hours and 2.7±1.0 hours after single and repeated doses, respectively.

Compound 2: Cialis®—Tadalafil (C₂₂H₁₉N₃O₄)

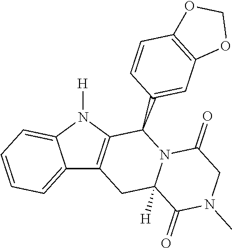 [SMILES: CN1CC(=O)N2[C@H](c3ccc4c(c3)OCO4)c3[nH]c4ccccc4c3C[C@@H]2C1=O]

### Mechanism of Action:

Tadalafil is a carboline derivative and PHOSPHODIESTERASE 5 INHIBITOR that is used primarily to treat ERECTILE DYSFUNCTION; BENIGN PROSTATIC HYPERPLASIA and PRIMARY PULMONARY HYPERTENSION.

Tadalafil is a Phosphodiesterase 5 Inhibitor. The mechanism of action of tadalafil is as a Phosphodiesterase 5 Inhibitor.

Tadalafil is a carboline-based compound with vasodilatory activity. Tadalafil selectively inhibits the cyclic guanosine monophosphate (cGMP)-specific type 5 phosphodiesterase- (PDE-5)-mediated degradation of cGMP, which is found in the smooth muscle of the corpus cavernosa and corpus spongiosum of the penis. Inhibition of cGMP degradation by tadalafil results in prolonged muscle relaxation, vasodilation, and blood engorgement of the corpus cavernosa, and, so, prolonged penile erection. An exemplary pack dose for 2 weeks is shown in Table 2.

TABLE 2

| Day | Cialis ®/Tandalafil (mg) | Famvir ®/Famciclovir (mg) |
|---|---|---|
| 1 | 10 mg | 500 + 500 + 500 + 1000 |
| 2 | 10 mg | 500 + 500 + 500 + 1000 |
| 3 | 10 mg | 500 + 500 + 500 + 1000 |
| 4 | 5 mg | 500 + 500 + 500 |
| 5 | 5 mg | 500 + 500 + 500 |
| 6 | 5 mg | 500 + 500 |
| 7 | 5 mg | 500 + 500 |
| 8 | 5 mg | 500 + 500 |
| 9 | 5 mg | 500 + 500 |
| 10 | 5 mg | 500 |
| 11 | 5 mg | 500 |
| 12 | 5 mg | 500 |
| 13 | 5 mg | 500 |
| 14 | 5 mg | 500 |

It can be appreciated that an aspect of the present technology can be a medicament for use in treating a viral infection. The medicament can comprise a combination composition including an anti-herpetic compound or a pharmaceutically acceptable salt thereof and an erectile dysfunction compound or a pharmaceutically acceptable salt thereof.

In some or all embodiments, the viral infection can be herpes or herpes simplex type II.

In some or all embodiments, the anti-herpetic compound can be selected from the group consisting of valacyclovir and famciclovir, or pharmaceutically acceptable salt thereof.

In some or all embodiments, the erectile dysfunction compound can be selected from the group consisting of sildenafil, tadalafil, avanafil and vardenafil, or pharmaceutically acceptable salt thereof.

In some or all embodiments, the combination composition can include 5 mg to 50 mg of the erectile dysfunction compound and 500 mg to 1000 mg of the anti-herpetic compound.

In some or all embodiments, the erectile dysfunction compound and the anti-herpetic compound can each be in the form of one or more solid dosage forms, tablets, capsules or in liquid form.

In some or all embodiments, the erectile dysfunction compound can be at a dose of 50 mg configured for use once a day for an initial 3 days of 14 days, and then at a dose of 25 mg configured for use once a day for a remaining 11 days of the 14 days.

In some or all embodiments, the anti-herpetic compound can be configured for use at least three times a day for an initial 3 days of 14 days, two times a day for a next 3 days of the 14 days, and once a day for a next 8 days of the 14 days.

In some or all embodiments, the anti-herpetic compound can be at a dose of 1000 mg configured for use three times a day for the initial 3 days of the 14 days, then at a dose of 1000 mg configured for use twice a day for the next 3 days of the 14 days, and then at a second dose of 1000 mg configured for use once a day for the next 8 days of the 14 days.

In some or all embodiments, the anti-herpetic compound can be valacyclovir and the erectile dysfunction compound is sildenafil.

In some or all embodiments, the erectile dysfunction compound can be at a dose of 10 mg of configured for use once a day for an initial 3 days of 14 days, and then at a dose of 5 mg configured for use once a day for a remaining 11 days of the 14 days.

In some or all embodiments, the anti-herpetic compound can be for use at least three four times a day for an initial 3 days of the 14 days, three times a day for a next 2 days of the 14 days, two times a day for a next 4 days of the 14 days, and once a day for a next 5 days of the 14 days.

In some or all embodiments, the anti-herpetic compound can be at a dose of 500 mg configured for use three times a day and at a dose of 1000 mg configured for use once a day on the initial 3 days of the 14 days. The anti-herpetic compound can be at a dose of 500 mg configured for use three times a day of the next 2 days of the 14 days. The anti-herpetic compound can be at a second dose of 500 mg configured for use twice a day on the next 4 days of the 14 days. The anti-herpetic compound can be at a third dose of 500 mg configured for use once a day on the next 5 days of the 14 days.

In some or all embodiments, the anti-herpetic compound can be famciclovir and the erectile dysfunction compound is tandalafil.

In some or all embodiments, the medicament can be presented as a pharmaceutical pack having the tablets or capsules of the anti-herpetic compound and the erectile dysfunction compound, the pack including instructions to a user to take one or more of the tablets or capsules for fourteen days.

Another aspect of the present technology can be a method of treating herpes in a patient in need thereof. The method can comprise a step or steps of administering to the patient an effective amount of a medicament comprising an anti-herpetic compound or a pharmaceutically acceptable salt thereof and an erectile dysfunction compound or a pharmaceutically acceptable salt thereof. The medicament can comprise a combination composition of approximately 5 mg to 50 mg of the erectile dysfunction compound and approximately 500 mg to 1000 mg of the anti-herpetic compound per dose.

In some or all embodiments, the anti-herpetic compound can be selected from the group consisting of valacyclovir and famciclovir, or pharmaceutically acceptable salt thereof. The erectile dysfunction compound can be selected from the group consisting of sildenafil, tadalafil, avanafil and vardenafil, or pharmaceutically acceptable salt thereof. The erectile dysfunction compound and the anti-herpetic compound can each be in the form of one or more solid dosage forms, tablets, capsules or in liquid form.

In some or all embodiments, the erectile dysfunction compound can be administered once a day for an initial 3 days of 14 days, and then can be administered once a day for a remaining 11 days of the 14 days. The anti-herpetic compound can be administered at least three times a day for the initial 3 days of the 14 days, two times a day for a next 3 days of the 14 days, and once a day for a next 8 days of the 14 days.

In some or all embodiments, the erectile dysfunction compound can be administered for the initial 3 days at a dose of 50 mg, and for the remaining 11 days at a dose of 25 mg. The anti-herpetic compound can be administered for the initial 3 days at a dose of 1000 mg, and for the next 3 days at a dose of 1000 mg, and for the next 8 days at a dose of 1000 mg.

In some or all embodiments, the erectile dysfunction compound can be administered once a day for an initial 3 days of 14 days, and then can be administered once a day for a remaining 11 days of the 14 days. The anti-herpetic compound can be administered at least four times a day for an initial 3 days of the 14 days, three times a day for a next 2 days of the 14 days, two times a day for a next 4 days of the 14 days, and once a day for a next 5 days of the 14 days.

In some or all embodiments, the erectile dysfunction compound can be administered for the initial 3 days at a dose of 10 mg, and for the remaining 11 days at a dose of 5 mg. The anti-herpetic compound can be administered for the initial 3 days at a dose of 500 mg for use three times a day and 1000 mg for use once a day, for the next 2 days at a dose of 500 mg, for the next 4 days at a dose of 500 mg, and for the next 5 days at a dose of 500 mg.

It can be appreciated that the above medicament composition can also include other compounds, additives, herbal extracts and/or carriers.

While embodiments of the medicament, compound, composition and method for treating genital herpes has been described in detail, it should be apparent that modifications and variations thereto are possible, all of which fall within the true spirit and scope of the invention. It can be appreciated the above described doses, amounts, regimen, protocol, and/or use, are exemplary and can include variants thereof.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

While some preferred embodiments of the invention have been described by way of example it should be appreciated that modifications and improvements can occur without departing from the scope of the appended claims.

What is claimed is:

1. A medicament for use in treating a viral infection, the medicament comprising a combination composition including an anti-herpetic compound or a pharmaceutically acceptable salt thereof and an erectile dysfunction compound or a pharmaceutically acceptable salt thereof.

2. The medicament according to claim 1, wherein the viral infection is herpes or herpes simplex type II.

3. The medicament according to claim 1, wherein the anti-herpetic compound is selected from the group consisting of valacyclovir and famciclovir, or pharmaceutically acceptable salt thereof, and wherein the erectile dysfunction compound is selected from the group consisting of sildenafil, tadalafil, avanafil and vardenafil, or pharmaceutically acceptable salt thereof.

4. The medicament according to claim 1, the combination composition including 5 mg to 50 mg of the erectile dysfunction compound and 500 mg to 1000 mg of the anti-herpetic compound.

5. The medicament according to claim 1, wherein the erectile dysfunction compound and the anti-herpetic compound are each in the form of one or more solid dosage forms, tablets, capsules or in liquid form.

6. The medicament according to claim 1, wherein the erectile dysfunction compound is at a dose of 50 mg configured for use once a day for an initial 3 days of 14 days, and then at a dose of 25 mg configured for use once a day for a remaining 11 days of the 14 days.

7. The medicament according to claim 1, wherein the anti-herpetic compound is configured for use at least three times a day for an initial 3 days of 14 days, two times a day for a next 3 days of the 14 days, and once a day for a next 8 days of the 14 days.

8. The medicament according to claim 7, wherein the anti-herpetic compound is at a dose of 1000 mg configured for use three times a day for the initial 3 days of the 14 days, then at a dose of 1000 mg configured for use twice a day for the next 3 days of the 14 days, and then at a second dose of 1000 mg configured for use once a day for the next 8 days of the 14 days.

9. The medicament according to claim 8, wherein the anti-herpetic compound is valacyclovir and the erectile dysfunction compound is sildenafil.

10. The medicament according to claim 5, wherein the erectile dysfunction compound is at a dose of 10 mg of configured for use once a day for an initial 3 days of 14 days, and then at a dose of 5 mg configured for use once a day for a remaining 11 days of the 14 days.

11. The medicament according to claim 10, wherein the anti-herpetic compound is for use at least three four times a day for an initial 3 days of the 14 days, three times a day for a next 2 days of the 14 days, two times a day for a next 4 days of the 14 days, and once a day for a next 5 days of the 14 days.

12. The medicament according to claim 11, wherein the anti-herpetic compound is at a dose of 500 mg configured for use three times a day and at a dose of 1000 mg configured for use once a day on the initial 3 days of the 14 days, wherein the anti-herpetic compound is at a dose of 500 mg configured for use three times a day of the next 2 days of the 14 days, wherein the anti-herpetic compound is at a second dose of 500 mg configured for use twice a day on the next 4 days of the 14 days, and wherein the anti-herpetic compound is at a third dose of 500 mg configured for use once a day on the next 5 days of the 14 days.

13. The medicament according to claim 12, wherein the anti-herpetic compound is famciclovir and the erectile dysfunction compound is tandalafil.

14. The medicament according to claim 1, wherein the medicament is presented as a pharmaceutical pack having the tablets or capsules of the anti-herpetic compound and the erectile dysfunction compound, the pack including instructions to a user to take one or more of the tablets or capsules for fourteen days.

15. A method of treating herpes in a patient in need thereof, said method comprising administering to the patient an effective amount of a medicament comprising an anti-herpetic compound or a pharmaceutically acceptable salt thereof and an erectile dysfunction compound or a pharmaceutically acceptable salt thereof, wherein the medicament comprises a combination composition of approximately 5 mg to 50 mg of the erectile dysfunction compound and approximately 500 mg to 1000 mg of the anti-herpetic compound per dose.

16. The method according to claim 15, wherein the anti-herpetic compound is selected from the group consisting of valacyclovir and famciclovir, or pharmaceutically acceptable salt thereof, and wherein the erectile dysfunction compound is selected from the group consisting of sildenafil, tadalafil, avanafil and vardenafil, or pharmaceutically acceptable salt thereof, and wherein the erectile dysfunction compound and the anti-herpetic compound are each in the form of one or more solid dosage forms, tablets, capsules or in liquid form.

17. The method according to claim 15, wherein the erectile dysfunction compound is administered once a day for an initial 3 days of 14 days, and then is administered once a day for a remaining 11 days of the 14 days, and wherein the anti-herpetic compound is administered at least three times a day for the initial 3 days of the 14 days, two times a day for a next 3 days of the 14 days, and once a day for a next 8 days of the 14 days.

18. The method according to claim 17, wherein the erectile dysfunction compound is administered for the initial 3 days at a dose of 50 mg, and for the remaining 11 days at a dose of 25 mg, and wherein the anti-herpetic compound is administered for the initial 3 days at a dose of 1000 mg, and for the next 3 days at a dose of 1000 mg, and for the next 8 days at a dose of 1000 mg.

19. The method according to claim 15, wherein the erectile dysfunction compound is administered once a day for an initial 3 days of 14 days, and then is administered once a day for a remaining 11 days of the 14 days, and wherein the anti-herpetic compound is administered at least four times a day for an initial 3 days of the 14 days, three times a day for a next 2 days of the 14 days, two times a day for a next 4 days of the 14 days, and once a day for a next 5 days of the 14 days.

20. The method according to claim 19, wherein the erectile dysfunction compound is administered for the initial 3 days at a dose of 10 mg, and for the remaining 11 days at a dose of 5 mg, and wherein the anti-herpetic compound is administered for the initial 3 days at a dose of 500 mg for use three times a day and 1000 mg for use once a day, for the next 2 days at a dose of 500 mg, for the next 4 days at a dose of 500 mg, and for the next 5 days at a dose of 500 mg.
